(19) United States
(12) Patent Application Publication
(10) Pub. No.: US 2021/0121484 A1
(43) Pub. Date: Apr. 29, 2021

(54) INHIBITORS OF ENPP1 AND METHODS OF USING SAME

(71) Applicant: The Regents of the University of California, Oakland, CA (US)

(72) Inventor: Arjun Deb, Los Angeles, CA (US)

(21) Appl. No.: 17/080,093

(22) Filed: Oct. 26, 2020

Related U.S. Application Data
(63) Continuation of application No. 16/193,352, filed on Nov. 16, 2018, now abandoned.
(60) Provisional application No. 62/587,684, filed on Nov. 17, 2017.

(51) Int. Cl. A61K 31/663; A61P 9/10; A61K 31/7076; A61P 19/04; A61K 31/216

(52) U.S. Cl. CPC A61K 31/663; A61P 9/10; A61K 31/216; A61P 19/04; A61K 31/7076

(57) ABSTRACT

Disclosed herein are methods for inhibiting ectopic calcification in soft tissues, such as heart tissue. Also provided herein are ectonucleotide pyrophosphatase/phosphodiesterase-1 (ENPP1) inhibitors.

### REFERENCE TO RELATED APPLICATIONS

This application claims the benefit of U.S. Provisional Patent Application No. 62/587,684, filed Nov. 17, 2017, the contents of which are fully incorporated by reference herein in their entirety.

### BACKGROUND

Mammalian tissues calcify with age and injury. Analogous to bone formation, osteogenic cells are thought to be recruited to the affected tissue and induce mineralization. Calcification of soft tissues is a cell mediated process that resembles bone formation in the skeletal system with calcification of the extracellular matrix by cells capable of mineralization. Pathological mineralization of soft tissues, or ectopic calcification, commonly occurs with tissue injury and degeneration and in common diseases such as diabetes and chronic kidney disease.

Calcification of the extracellular matrix is critically regulated by the balance of extracellular phosphate (Pi) and pyrophosphate (PPi). Pyrophosphate is generated at the cell surface by the enzyme ectonucleotide pyrophosphatase/phosphodiesterase-1 (ENPP1) that breaks down ATP to AMP and PPi. Pyrophosphate promotes mineralization by serving as a substrate for tissue non-specific alkaline phosphatase that hydrolyzes pyrophosphate to generate inorganic phosphate. Thus, inhibition of ENPPI reduces the amount of PPi formed and subsequent calcification.

As there are currently no drugs available to retard calcification in soft tissues, blood vessels or valves, a significant unmet clinical need exists for identifying agents that can inhibit pathological calcification of tissues.

### SUMMARY

Disclosed herein is a method of treating or preventing ectopic calcification, such as within heart tissue, in a subject, comprising administering a compound selected from rosmarinic acid, ARL67156, and etidronic acid, or a pharmaceutically acceptable salt and/or prodrug of any of the foregoing.

In certain embodiments, the subject has a disease, disorder or condition selected from diabetes, kidney disease, and myocardial injury associated with ischemia or inflammation. In certain embodiments, the subject has heart disease. In certain embodiments, the subject has pseudoxanthoma elasticum.

In certain embodiments, the present invention provides a pharmaceutical preparation suitable for use in a human patient in the treatment or prevention of treating or preventing ectopic calcification, such as within heart tissue, comprising an effective amount of any of the compounds described herein and one or more pharmaceutically acceptable excipients. In certain embodiments, the pharmaceutical preparations may be for use in treating or preventing a condition or disease as described herein.

### BRIEF DESCRIPTION OF THE DRAWINGS

FIG. 1 shows expression of ENPP1 by qPCR in injured and uninjured regions of hearts of B6 and C3H mice 7 days after cryo injury (mean±S.E.M., n=6, *p<0.01).

### DETAILED DESCRIPTION

Calcification of soft tissues is a cell mediated process that resembles bone formation in the skeletal system with calcification of the extracellular matrix by cells capable of mineralization. Analogous to bone formation, osteogenic cells are thought to be recruited to the affected tissue and induce mineralization. Pathological mineralization of soft tissues, or ectopic calcification, commonly occurs with tissue injury and degeneration and in common diseases such as diabetes and chronic kidney disease.

In the heart, calcification of cardiac muscle leads to conduction system disturbances and is one of the most common pathologies underlying heart blocks. Calcification of the cardiovascular system is associated with more than 100-500 fold increase in cardiovascular mortality. Myocardial calcification is observed in the aging heart and in patients with diabetes, renal disease, and myocardial injury secondary to ischemia or inflammation. Cardiac pump dysfunction and arrhythmias can also occur depending on the extent and anatomic site of calcification and calcified myocardial scars have been reported to cause refractory ventricular tachycardia. Cardiac calcification is also a prognostic indicator of poor outcomes following myocardial infarction or myocarditis.

Cardiac fibroblasts can act as osteoblasts in forming calcifications in the heart, such as by contributing to mineralization of the extracellular matrix around the heart.

Calcification of the extracellular matrix is critically regulated by the balance of extracellular phosphate (Pi) and pyrophosphate (PPi). Pyrophosphate is generated at the cell surface by the enzyme ectonucleotide pyrophosphatase/phosphodiesterase-1 (ENPP1) that breaks down ATP to AMP and PPi. ENPP1 is expressed in osteoblasts, regulating bone mineralization. For instance, pyrophosphate promotes mineralization by serving as a substrate for tissue non-specific alkaline phosphatase that hydrolyzes pyrophosphate to generate phosphate moieties that can precipitate with calcium to form calcium hydroxyapatite. Thus, increased levels of ENPP1 generate PPi that serves as a substrate for formation of ectopic calcium deposits.

The disclosed methods provide inhibitors of ENPP1, which substantially attenuate ectopic calcification in heart tissues, including heart valves. The exemplification in Appendix A reflects the potency of the disclosed ENPP1 inhibitors to disrupt pathological calcification regardless of the disease, disorder or condition that led to its formation.

Several ENPP1 inhibitors are known in the art. For example, rosmarinic acid (also known as SYL-001) has the following structure:

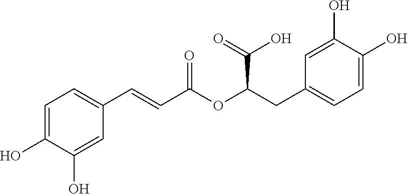 [SMILES: O=C(/C=C/c1ccc(O)c(O)c1)O[C@H](Cc1ccc(O)c(O)c1)C(=O)O]

and is known for its activity as an anti-oxidant and GABA transaminase inhibitor. (See, Sassi, et al. J. Clin. Invest. 2014 124:5385-5397.) Another ENPP1 inhibitor is ARL67156, which has the following structure:

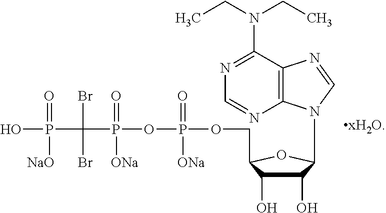

Its ENPP inhibitory activity has been described by Cote et al. (Eur. J. Pharmacol. 2012 689:139-146) and Levesque et al. (Br. J. Pharmacol. 2007 152:141-150). A third compound with ENPP1 inhibitory activity is a bisphosphonate known as etidronic acid:

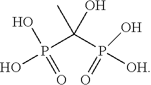

Although primarily used for their anti-resorptive effect on bone, first generation bisphosphonates such as etidronic acid can bind to calcium hydroxyapatite in sites of active bone remodeling and, as they are not hydrolyzable, prevent further bone mineralization. It is also an antagonist to vascular mineralization.

Disclosed herein is a method of treating or preventing ectopic calcification, such as within heart tissue, in a subject, comprising administering a compound selected from rosmarinic acid, ARL67156, and etidronic acid or a pharmaceutically acceptable salt and/or prodrug of any of the foregoing. In certain embodiments, the compound is rosmarinic acid or a pharmaceutically acceptable salt and/or prodrug thereof. In certain embodiments, the compound is ARL67156 or a pharmaceutically acceptable salt and/or prodrug thereof. In certain embodiments, the compound is etidronic acid or a pharmaceutically acceptable salt and/or prodrug thereof.

In certain embodiments, one or more disclosed compounds can be administered to the subject. For example, rosmarinic acid and ARL67156 may be conjointly administered to the subject. In certain embodiments, rosmarinic acid and etidronic acid, or another bisphosphonate, can be conjointly administered to the subject. In some embodiments, ARL67156 and etidronic acid, or another bisphosphonate can be conjointly administered to the subject.

In certain embodiments, the bisphosphonate can be non-nitrogenous, such as clondrate and tiludronate. In certain embodiments, the bisphosphonate can be nitrogenous, such as pamidronate, neridronate, olpadronate, alendronate, ibandronate, risedronate, and zoledronate.

In certain embodiments, the therapeutic may be a prodrug of rosmarinic acid, ARL67156, or etidronic acid, e.g., wherein a hydroxyl in the parent compound is presented as an ester or a carbonate, a phosphate or phosphonic acid is presented as an ester or amide derivative, or a carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

In certain embodiments, the subject has a disease, disorder or condition selected from diabetes, kidney disease, and myocardial injury associated with ischemia or inflammation. In certain embodiments, the subject has heart disease or vascular disease. In certain embodiments, the subject has pseudoxanthoma elasticum (PXE). PXE is characterized by progressive calcification of soft tissues. No effective treatments for this disease are known, and individuals die from progressive calcification of vital organs.

Due to aging or injury, all of these diseases and disorders can be accompanied by mineralization, either at the site of damage or throughout the organ, such as the heart. Disclosed herein are methods of treating ectopic calcification associated with organ injury in a subject, comprising administering a compound selected from rosmarinic acid, ARL67156, and etidronic acid.

### Definitions

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into therapeutically active agents. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) and esters or amides of phosphates and phosphonic acids are preferred prodrugs of the present invention.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Example

Cardiac fibroblasts adopt osteogenic cell fates and contribute to pathologic heart calcification. (Adapted from Cell Stem Cell (2017) 20:1-15, which is incorporated by reference herein in its entirety, and in particular for the experiments, results, FIGURES, and diagrams described therein.)

### Summary

Mammalian tissues calcify with age and injury. Ectopic calcification of soft tissues is thought to be a dynamic cell mediated process analogous to bone formation in the skeletal system, in which bone forming cells are recruited to the affected tissue and induce mineralization of the matrix. In the heart, calcification of heart muscle leads to conduction system disturbances and is one of the most common pathologies underlying heart blocks. Yet the phenotype of the cell contributing to and the mechanisms regulating cardiac calcification remain unknown. In this report, we investigate the identity of the cell and target mechanisms that contribute to pathologic heart muscle calcification. Using genetic fate map techniques, murine models of heart calcification and in vivo cell transplantation assays, we show that the cardiac fibroblast adopts an osteoblast cell-like fate and contributes directly to heart muscle calcification. ENPP1 an enzyme that generates pyrophosphate and promotes formation of hydroxyapatite is induced in cardiac fibroblasts after injury. Inhibition of ENPP1 with small molecules significantly attenuated cardiac calcification and inhibitors of bone mineralization completely prevented ectopic cardiac calcification and led to better post injury cardiac function. Taken together, these findings highlight the plasticity of fibroblasts in contributing to ectopic calcification and identify pharmacological targets for treating cardiac calcification.

### Results

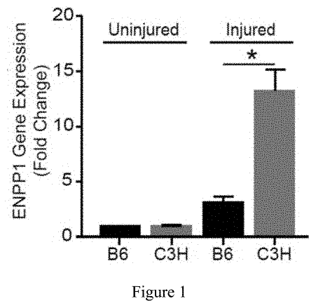

To test this hypothesis, we subjected cardiac fibroblasts (isolated from 8 week old, male and female C57BL/6J murine hearts) to osteogenic differentiation. Osteogenic differentiation was induced by treating isolated fibroblasts with culture medium known to induce osteogenic differentiation of mesenchymal stem cells (Jaiswal et al., 1997). Treatment of cardiac fibroblasts with osteogenic differentiation medium (DM) for 21 days led to deposition of calcium hydroxyapatite, visualized with Alizarin Red staining (Cell Stem Cell (2017) 20:1-15, FIG. 1B). No calcium deposition occurred in cardiac fibroblasts treated with control growth medium for the same duration (Cell Stem Cell (2017) 20:1-15, FIG. 1A). Gene expression analysis by RNA sequencing of cardiac fibroblasts harvested at different time points following induction of differentiation revealed clusters of genes whose expression was significantly altered in a temporal specific manner (Cell Stem Cell (2017) 20:1-15, FIG. 1C). Genes regulating cell cycle that were highly expressed in undifferentiated cardiac fibroblasts were down-regulated at the onset of differentiation and remained at low expression levels throughout the duration of osteogenic differentiation, consistent with the principle that induction of differentiation is associated with reduced rates of proliferation (Buttitta and Edgar, 2007) (Cell Stem Cell (2017) 20:1-15, FIG. 1C; Table 1).

TABLE 1
List of families of genes upregulated in cardiac fibroblasts treated with osteogenic differentiation
medium. Principal families of genes downregulated (green) or upregulated (blue, magenta,
orange) in cardiac fibroblasts subjected to osteogenic differentiation compared to controls.
(Count: the number of differentially expressed genes that overlap with genes associated
with the biological term or gene family. List total: the total number of annotated differentially
expressed genes within the category or ontology. Pop Hits: the number of genes within
the ontology based category associated with the term. Pop Total: the total number of
genes from the category. FDR: the false discovery rate)

| | | | | List | Pop | Pop | |
|---|---|---|---|---|---|---|---|
| Category | Cluster | Terms | Count | Total | Hits | Total | FDR |
| SP_PIR_KEYWORDS | Blue | disulfide | 157 | 402 | 2469 | 17854 | 1.57E−33 |
| | | bond | | | | | |
| GOTERM_CC | Blue | extracellular | 69 | 327 | 774 | 12504 | 2.10E−16 |
| | | region part | | | | | |
| GOTERM_MF | Blue | polysaccharide | 17 | 309 | 128 | 13288 | 6.07E−05 |
| | | binding | | | | | |
| GOTERM_BP | Blue | immune | 52 | 340 | 471 | 13588 | 5.46E−16 |
| | | response | | | | | |
| GOTERM_BP | Blue | locomotory | 24 | 340 | 239 | 13588 | 5.52E−05 |
| | | behavior | | | | | |
| GOTERM_BP | Blue | regulation | 17 | 340 | 139 | 13588 | 6.45E−04 |
| | | of cytokine | | | | | |
| | | production | | | | | |
| SP_PIR_KEYWORDS | Magenta | signal | 30 | 82 | 2970 | 17854 | 2.80E−02 |
| SP_PIR_KEYWORDS | Orange | signal | 119 | 351 | 2970 | 17854 | 3.75E−12 |
| SP_PIR_KEYWORDS | Orange | gpi-anchor | 14 | 351 | 124 | 17854 | 1.00E−03 |
| GOTERM_BP | Orange | defense | 26 | 273 | 448 | 13588 | 6.27E−03 |
| | | response | | | | | |
| KEGG_PATHWAY | Orange | drug | 13 | 115 | 75 | 5738 | 2.15E−05 |
| | | metabolism | | | | | |
| GOTERM_BP | Green | cell cycle | 101 | 440 | 611 | 13588 | 1.42E−40 |
| GOTERM_CC | Green | chromosome, | 33 | 394 | 111 | 12504 | 2.29E−19 |
| | | centromeric | | | | | |
| | | region | | | | | |
| GOTERM_CC | Green | intracellular | 122 | 394 | 1919 | 12504 | 4.77E−12 |
| | | non- | | | | | |
| | | membrane- | | | | | |
| | | bounded | | | | | |
| | | organelle | | | | | |
| UP_SEQ_FEATURE | Green | nucleotide | 71 | 522 | 907 | 16021 | 1.90E−08 |
| | | phosphate- | | | | | |
| | | binding | | | | | |
| | | region: ATP | | | | | |
| GOTERM_BP | Green | microtubule- | 33 | 440 | 211 | 13588 | 4.13E−10 |
| | | based | | | | | |
| | | process | | | | | |
| GOTERM_BP | Green | cell | 49 | 440 | 561 | 13588 | 1.06E−06 |
| | | adhesion | | | | | |
| GOTERM_BP | Green | DNA | 45 | 440 | 421 | 13588 | 8.87E−09 |
| | | metabolic | | | | | |
| | | process | | | | | |
| SP_PIR_KEYWORDS | Green | dna | 20 | 542 | 85 | 17854 | 9.28E−09 |
| | | replication | | | | | |
| GOTERM_BP | Green | meiosis | 14 | 440 | 88 | 13588 | 7.88E−03 |
| GOTERM MF | Green | cytoskeletal | 32 | 402 | 414 | 13288 | 4.42E−03 |
| | | protein binding | | | | | |
| SP_PIR_KEYWORDS | Green | extracellular | 23 | 542 | 213 | 17854 | 7.73E−04 |
| | | matrix | | | | | |

In contrast, genes that were minimally expressed in cardiac fibroblasts were induced in a specific temporal manner during the course of osteogenic differentiation (Cell Stem Cell (2017) 20:1-15, FIG. 1C) and included sets of genes known to regulate inflammation, extracellular matrix proteins and cell metabolism (Table 1). Next, we created an osteogenic signature based on a set of 37 genes that are induced during osteogenic differentiation (Chen et al., 2012); (Choi et al., 2010); (Graneli et al., 2014); (Harkness et al., 2011); (Hoshiba et al., 2009); (Liu et al., 2013); (Miguez et al., 2014); (Nora et al., 2012); (Olivares-Navarrete et al., 2011); (Cell Stem Cell (2017) 20:1-15, Figure S1B). We used the mean fold change in expression of this set of genes to quantitatively determine an osteogenic signature and observed that compared to control cardiac fibroblasts, cardiac fibroblasts subjected to osteogenic differentiation progressively adopted an osteogenic signature (Cell Stem Cell (2017) 20:1-15, FIG. 1D). Quantitative PCR confirmed induction of expression of canonical osteoblast genes (Runx2, osteocalcin, osterix, bone sialoprotein and osteopontin) in cardiac fibroblasts following osteogenic differentiation (Cell Stem Cell (2017) 20:1-15, FIG. 1 E-I). We next performed experiments with a control cell such as an endothelial cell to determine whether the ability to undergo osteogenic differentiation and induce mineralization is specific to fibroblasts. We treated human arterial endothelial cells (HAECs) (Romanoski et al., 2010) and human cardiac fibroblasts to osteogenic DM in vitro for 21 days (Cell Stem Cell (2017) 20:1-15, Figure S2A-D). Similarly to murine cardiac fibroblasts, human cardiac fibroblasts robustly induced mineralization of the matrix (Cell Stem Cell (2017) 20:1-15, Figure S2B,D) but HAECs under identical conditions failed to induce mineralization of the matrix (Cell Stem Cell (2017) 20:1-15, Figure S2A,C) suggesting that the ability to undergo osteogenic differentiation was not autonomous of the phenotype of the cell. We next investigated whether the changes in expression of osteogenic genes in cardiac fibroblasts was reversible. We treated cardiac fibroblasts with osteogenic DM for 14 days and then reseeded them in the presence or absence of osteogenic DM for another 14 days (Cell Stem Cell (2017) 20:1-15, Figure S2E). Expression of the canonical master osteogenic transcription factor Runx2 did not substantially change upon removing the cells from an osteogenic environment and placing them under regular growth conditions (Cell Stem Cell (2017) 20:1-15, Figure S2F). These observations suggest that the osteogenic phenotype adopted by cardiac fibroblasts is stable.

To confirm the observations that cardiac fibroblasts undergo osteogenic differentiation, we isolated cardiac fibroblasts from transgenic mice in which cardiac fibroblasts are genetically labeled. For this purpose we crossed transgenic mice harboring a tamoxifen inducible Cre recombinase driven by enhancer elements of the Type 1 collagen α2 gene (Col1a2-CreERT) or a Cre recombinase driven by promoter elements of the Fibroblast specific protein 1 gene (FSP1-Cre) to the lineage reporter R26R^{tdTomato} mice to create progeny Col1a2-CreERT:R26R^{tdTomato} or FSP1-Cre:R26R^{tdTomato} mice (Qian et al., 2012); (Ubil et al., 2014); (Zheng et al., 2002). We have recently shown that administration of tamoxifen for 10 days in Col1a2-CreERT:R26R^{tdTomato} mice results in specific labeling of approximately 55% of cardiac fibroblasts (Ubil et al., 2014). We isolated tdTomato labeled cardiac fibroblasts from Col1a2-CreERT:R26R^{tdTomato} mice (99% purity by flow cytometry) (Cell Stem Cell (2017) 20:1-15, Figure S3A,B), subjected labeled cardiac fibroblasts to osteogenic differentiation (Cell Stem Cell (2017) 20:1-15, FIG. 1J,K) and observed calcium hydroxyapatite deposition (Cell Stem Cell (2017) 20:1-15, FIG. 1K) but not in labeled cardiac fibroblasts cultured under control conditions (Cell Stem Cell (2017) 20:1-15, FIG. 1J). In the FSP1-Cre:R26R^{tdTomato} transgenic mice, the FSP-1 promoter elements drive Cre recombinase and this system has been used to track fibroblast fates (Qian et al., 2012); (Song et al., 2012). Labeled cardiac fibroblasts were isolated from FSP1-Cre:R26R^{tdTomato} mice by flow cytometry (98% purity) (Cell Stem Cell (2017) 20:1-15, Figure S3C,D) and upon induction of osteogenic differentiation (Cell Stem Cell (2017) 20:1-15, FIG. 1L,M) formed calcium hydroxyapatite (Cell Stem Cell (2017) 20:1-15, FIG. 1M) while FSP1 labeled cardiac fibroblasts under control conditions did not (Cell Stem Cell (2017) 20:1-15, FIG. 1L). The extent of hydroxyapatite deposition following 21 days of osteogenic differentiation was not significantly different between FSP1 and Col1a2 labeled cardiac fibroblasts (Cell Stem Cell (2017) 20:1-15, FIG. 1N) suggesting that the ability to undergo osteogenic differentiation was not dependent on the Cre drivers chosen.

We next investigated the possibility that osteogenic differentiation of genetically labeled cardiac fibroblasts could be secondary to the presence of progenitor like cells that undergo osteogenic differentiation. We first determined expression of the progenitor marker C-Kit in FSP1 labeled cardiac fibroblasts but observed that 99.9% of labeled cells were negative for C-Kit expression (Cell Stem Cell (2017) 20:1-15, Figure S3E). Cardiac progenitors identified by expression of Stem cell antigen (Sca-1) are the most populous type of progenitor cell present within the mouse heart (Leri et al., 2005). More recently, a colony forming unit-fibroblast (CFU-F) has been identified in the heart to mark cardiac stromal cells with progenitor characteristics and also expresses Sca-1 (Chong et al., 2011). We isolated labeled cardiac fibroblasts from FSP-1Cre:R26R^{tdTomato} mice and separated the tdTomato labeled cells into a predominantly Sca-1 expressing and Sca-1 negative population (98.5% and 97% purity respectively) by flow cytometry (Cell Stem Cell (2017) 20:1-15, Figure S3F,G). Consistent with CFU properties of Sca-1 expressing cells (Chong et al., 2011), we observed significant reduction of colony forming unit capacity of tdTomato(+)Sca-1(−) fraction compared to tdTomato(+)Sca-1(+) fractions (Cell Stem Cell (2017) 20:1-15, Figure S3H,I). However, upon osteogenic differentiation, there was no significant difference in the extent of calcium hydroxyapatite deposition between tdTomato(+)Sca-1(+) and tdTomato(+)Sca-1(−) cells (Cell Stem Cell (2017) 20:1-15, FIG. 1O,P), thereby suggesting that osteogenic differentiation of genetically labeled fibroblasts is unlikely to be secondary to the presence of Sca-1 expressing progenitor cells.

Pericytes in organs are thought to possess multipotent progenitor cell characteristics (Crisan et al., 2008) and we next determined whether pericytes potentially present in the genetically labeled fibroblast pool could have contributed to calcification. Pericytes can be identified by expression of NG2, CD146 and Platelet Derived Growth Factor Receptorβ (PDGFRβ) (Murray et al., 2016). We examined sections of uninjured hearts of FSP1Cre:R26R^{tdTomato} mice but observed minimal expression of NG2 (98.4% of tdTomato cells negative for NG2) or CD146 (99.2% of tdTomato cells negative for NG2) in tdTomato labeled cells (Cell Stem Cell (2017) 20:1-15, Figure S4A-F). With flow cytometry, we did observe a fraction of FSP1 labeled cells to express PDGFRβ and separated the cells into tdTomato(+)PDGFRβ enriched and tdTomato(+)PDGFRβ depleted cells (Cell Stem Cell (2017) 20:1-15, Figure S4G,H). Upon osteogenic differentiation, the extent of hydroxyapatite deposition was similar in FSP1 labeled PDGFRβ enriched and FSP labeled PDGFRβ depleted pools (Cell Stem Cell (2017) 20:1-15, FIG. 1Q,R) thus demonstrating that PDGFRβ expressing cells are not the predominant source of cells in the fibroblast pool undergoing osteogenic differentiation. Taken together these observations suggest that cardiac fibroblasts isolated from the adult murine heart can adopt osteogenic cell like fates and contribute to calcium deposition in vitro.

We next investigated whether cardiac fibroblasts can adopt osteoblast cell like fates in vivo and directly contribute to ectopic calcification of the myocardium. To address this question, we created three murine models of myocardial calcification and determined with lineage trace techniques whether genetically labeled cardiac fibroblasts adopted an osteoblast phenotype and contributed to heart calcification in vivo. Cardiac injury or aging in certain strains of mice (e.g. C3H/HeJ, BALB/cByJ, DBA/2J) can lead to the development of calcification within the myocardium (Glass et al., 2013); (Ivandic et al., 1996); (Korff et al., 2006). We used several different methods to induce myocardial injury in C3H strain of mice. First, we administered high dose systemic steroids daily for 10 days which is known to induce myocyte necrosis (Sparks et al., 1955). Uninjured hearts did not exhibit any calcification (Cell Stem Cell (2017) 20:1-15, FIG. 2A,B) but animals injected with steroids exhibited patchy cardiac calcification within 5 days of cessation of steroid injections (Cell Stem Cell (2017) 20:1-15, FIG. 2C,D). Cryo-probe mediated injury of the mid ventricle (Aherrahrou et al., 2004) also resulted in calcification of the injury region within 7 days of injury (Cell Stem Cell (2017) 20:1-15, FIG. 2E,F). Finally, ischemic injury of the myocardium by ligating the left anterior descending (LAD) coronary artery, (Korff et al., 2006) led to patchy calcification of the injury region within 4 weeks of ischemic insult (Cell Stem Cell (2017) 20:1-15, FIG. 2G,H). Consistent with the known association of fibrosis and calcification, we observed calcification only in regions where there was fibrosis (identified by Masson trichrome staining) (Cell Stem Cell (2017) 20:1-15, FIG. 2 I,J).

Next we induced injury in the Col1a2-CreERT:R26R^{tdTomato} transgenic mice to determine whether fibroblasts adopted an osteoblast fate and contributed to myocardial calcification. For this purpose the Col1a2-CreERT:R26R^{tdTomato} mice (B6 background) were backcrossed to a C3H background for 8-10 generations to obtain a robust calcification phenotype after heart injury. Tamoxifen was administered for 10 days to 8 week old animals to label cardiac fibroblasts and following a 5 day gap, myocardial injury was induced either with hydrocortisone, cryo-probe or permanent ligation of the LAD coronary artery and tissue harvested at 5 days following completion of hydrocortisone injections, or 7 days and 4 weeks after cryo and ischemic injury respectively (Cell Stem Cell (2017) 20:1-15, FIG. 2K). In uninjured hearts of Col1a2-CreERT:R26R^{tdTomato} mice, tdTomato labeled cardiac fibroblasts did not express the canonical osteogenic markers Runx2, Osteocalcin (OCN) or Osterix (Cell Stem Cell (2017) 20:1-15, FIG. 2 L-N). In the model of cardiac calcification induced by systemic high dose steroids, tdTomato labeled cardiac fibroblasts expressed the master osteogenic transcription factor Runx2 and osteoblast markers osteocalcin and osterix and were arranged in close physical apposition to calcium hydroxyapatite deposits (Cell Stem Cell (2017) 20:1-15, FIG. 2 O-T). The extracellular matrix protein osteopontin has been implicated in the regulation of ectopic cardiac calcification and we observed abundant osteopontin expression in labeled fibroblasts adjacent to calcified myocardium (Cell Stem Cell (2017) 20:1-15, Figure S5A-E). We analyzed the expression of osteogenic markers by tdTomato labeled cardiac fibroblasts within the region of calcification and observed that 23.5±3.6%, 35.9±4.3% and 37.9±9.4% (mean±S.E.M.) of labeled cardiac fibroblasts expressed the markers Runx2, OCN and Osterix respectively while the fraction of labeled cardiac fibroblasts expressing these markers in uninjured hearts was less than 1.5% (p<0.05) (Cell Stem Cell (2017) 20:1-15, FIG. 2U). In cryo and ischemic injury induced myocardial calcification we similarly observed a substantial fraction of labeled cardiac fibroblasts to express osteogenic markers. The fraction of labeled cardiac fibroblasts expressing osteogenic markers (Runx2, OCN) was approximately 42.7±3% and 53.6±3.4% for cryo injury, (mean±S.E.M.) (Cell Stem Cell (2017) 20:1-15, FIG. 3A-F) and 43.5±1.7% and 58.4±10.9% for ischemic injury (mean±S.E.M.) (Cell Stem Cell (2017) 20:1-15, FIG. 3G-K) respectively with expression of these markers in the control uninjured hearts at less than 1% of labeled fibroblasts (p<0.05) (Cell Stem Cell (2017) 20:1-15, FIG. 3F,K). The number of Runx2 positive cells not labeled by tdTomato was approximately 38% after injury, that could reflect limitations with efficiency of Cre labeling or unlabeled fibroblasts expressing osteogenic markers.

To corroborate our findings with the Col1a2-CreERT mice, we used the TCF21MerCreMer:R26R^{tdTomato} mouse (backcrossed to a C3H background), that has been used to specifically label cardiac fibroblasts in the adult heart (Acharya et al., 2011); (Kanisicak et al., 2016). In uninjured hearts injected with tamoxifen, tdTomato labeled cells did not express Runx2 or OCN (Cell Stem Cell (2017) 20:1-15, FIG. 4A,B). However following cryo injury, we observed that substantial numbers of tdTomato labeled cardiac fibroblasts in the region of injury expressed osteogenic markers (Cell Stem Cell (2017) 20:1-15, FIG. 4C-E), corroborating our findings with the Col1a2-CreERT driver. As calcification occurs in the region of injury, we also performed immunostaining to determine expression of Runx2 in myocytes but did not observe any myocytes expressing Runx2 (Cell Stem Cell (2017) 20:1-15, Figure S5F-H). We also did not observe any evidence of intravascular calcification (Cell Stem Cell (2017) 20:1-15, Figure S5I-K). Finally, in strains of mice (B6) that do not exhibit calcification after injury, labeled cardiac fibroblasts did not express any osteogenic markers demonstrating that expression of osteoblast markers in cardiac fibroblasts is not simply a response to injury but is associated with the calcific phenotype (Cell Stem Cell (2017) 20:1-15, Figure S5L,M). Collectively these in vivo experiments using fate mapping with independent Cre drivers and multiple models of myocardial calcification suggest that cardiac fibroblasts can adopt an osteoblast cell like fate.

Osteoblasts not only express extracellular matrix proteins but also directly contribute to the mineralization of the extracellular matrix. We next investigated whether cardiac fibroblasts in regions of myocardial calcification can directly contribute to mineralization of extracellular matrix. To address this question, we dissected regions of myocardial calcification following cryo-injury in Col1a2-CreERT:R26R^{tdTomato} mice and performed in vitro explant culture of calcified myocardial tissue (Cell Stem Cell (2017) 20:1-15, FIG. 5A). We observed that tdTomato labeled cardiac fibroblasts migrated outwards from the control or calcified myocardial tissue (Cell Stem Cell (2017) 20:1-15, FIG. 5B). Immunofluorescent staining showed that 24.8±2.9% and 64.7±3% (mean±S.E.M.) of tdTomato labeled cardiac fibroblasts that had migrated from the calcific explant culture expressed Runx2 and OCN (Cell Stem Cell (2017) 20:1-15, FIG. 5C-E). In contrast, tdTomato labeled cardiac fibroblasts migrating from explanted myocardial cultures of non-injured hearts did not express osteogenic markers (Cell Stem Cell (2017) 20:1-15, FIG. 5E). Labeled fibroblasts (tdTomato+) from the control or calcified myocardial explanted tissue were then sorted by flow cytometry to 99% purity, and injected into subcutaneous pockets surgically fashioned on the dorsum of mice (Abdallah et al., 2008) (recipient mice were wild type C3H strain, no tdTomato transgene present) (Cell Stem Cell (2017) 20:1-15, FIG. 5F). As a control, we isolated cardiac fibroblasts from explant cultures of uninjured heart tissue of Col1a2-CreERT:R26R^{tdTomato} mice and implanted them in an identical manner in a subcutaneous pocket fashioned on the contralateral side of the same animal (Cell Stem Cell (2017) 20:1-15, FIG. 5G). Finally another subcutaneous dorsal pocket was created to inject medium without any cells (Cell Stem Cell (2017) 20:1-15, FIGS. 5A,G). We subjected the animals to Computer Associated Tomography (micro-CT) at weekly intervals. At 4 weeks after implantation, we observed a significantly greater degree of calcification of the subcutaneous region injected with labeled cardiac fibroblasts isolated from calcific myocardial tissue compared to subcutaneous tissue injected with labeled fibroblasts from uninjured animals or not injected with fibroblasts (Cell Stem Cell (2017) 20:1-15, FIG. 5H-J). There was no difference in the degree of calcification between subcutaneous pockets injected without cells or with tdTomato labeled cells isolated from explant culture of uninjured myocardium (Cell Stem Cell (2017) 20:1-15, FIG. 5J). To confirm that the increase in calcification represented new osteogenic activity, we performed positron emission tomography (micro-PET) with ¹⁸NaF radionuclide, which binds to calcium hydroxyapatite in newly formed bone and is used in clinical practice to identify regions of new bone formation (Czernin et al., 2010). We observed a significant and marked increase in PET signal in subcutaneous tissues injected with labeled cardiac fibroblasts isolated from calcified myocardial tissue compared to control groups (Cell Stem Cell (2017) 20:1-15, FIG. 5K,L) and the anatomic location of the enhanced signal colocalized with region of subcutaneous calcification noted on the CT scan (Cell Stem Cell (2017) 20:1-15, FIG. 5L).

We next dissected the calcified subcutaneous tissue to determine the presence of tdTomato labeled osteogenic cells. Histological stains (Von Kossa and Hematoxylin) identified subcutaneous calcific deposits (Cell Stem Cell (2017) 20:1-15, FIG. 5M,N). On immunofluorescent staining, we observed abundant tdTomato labeled cells expressing osteogenic markers OCN and Runx2 and present on the edges of calcified matrix (Cell Stem Cell (2017) 20:1-15, FIG. 50-T). These cell transplantation experiments demonstrate that cardiac fibroblasts harvested from calcific but not uninjured myocardium, when injected into soft tissues are sufficient to induce ectopic soft tissue calcification, showing a direct role of the cardiac fibroblast in mediating soft tissue calcification.

Having demonstrated that cardiac fibroblasts can induce mineralization of the matrix, we investigated mechanisms of osteogenesis or mineralization that could be potentially targeted to decrease ectopic calcification. We performed RNA-seq on uninjured and injured cardiac regions of C3H (calcify after injury) and B6 strains (no calcification after injury) (Cell Stem Cell (2017) 20:1-15, Figure S6A). Gene expression analysis demonstrated that calcific hearts compared to non-calcific hearts responded to injury with a dramatically different transcriptional program. In contrast to only 70 odd genes that were differentially upregulated following injury in non-calcified mouse hearts (B6) about 960 genes were upregulated in C3H hearts following injury induced calcification (Cell Stem Cell (2017) 20:1-15, Figure S6B). Out of the 960 differentially upregulated genes, only 35 were found to be common or upregulated in both C3H and B6 hearts after injury (Cell Stem Cell (2017) 20:1-15, Figure S6B) illustrating of the overlapping but dramatically different magnitude of the injury response. Families of genes regulating diverse aspects of an injury response including inflammation, extracellular matrix proteins, cell proliferation and collagen production were differentially expressed between the calcific and non-calcific hearts after injury (Table 2).

TABLE 2
List of differentially upregulated genes in both C3H and B6 injured heart regions,
arranged in clusters according to DAVID functional annotation of genes. The highest
rank biological terms from each cluster analysis is reported with a maximum false
discovery rate (FDR) of 0.05. (Count: the number of induced genes that overlap with
genes associated with the biological term or gene family. List total: the total number
of annotated induced genes within the category or ontology. Pop Hits: the number
of genes within the ontology based category associated with the term. Pop Total:
the total number of genes from the category. FDR: the false discovery rate)

| | | Terms: | | | | | |
|---|---|---|---|---|---|---|---|
| | | Increased | | List | Pop | Pop | |
| Category | Strain | Expression | Count | Total | Hits | Total | FDR |
| SP_PIR_KEYWORDS | B6 | signal | 31 | 66 | 2970 | 17854 | 2.93E−05 |
| GOTERM_MF | B6 | carbohydrate | 12 | 43 | 317 | 13288 | 2.86E−06 |
| | | binding | | | | | |
| GOTERM BP | B6 | cell adhesion | 11 | 41 | 561 | 13588 | 5.01E−03 |
| GOTERM_CC | B6 | extracellular | 23 | 55 | 1680 | 12504 | 6.36E−04 |
| | | region | | | | | |
| SP_PIR_KEYWORDS | C3H | glycoprotein | 392 | 824 | 3600 | 17854 | 4.73E−70 |
| GOTERM_CC | C3H | lysosome | 33 | 655 | 178 | 12504 | 9.14E−07 |
| INTERPRO | C3H | Immunoglobulin | 55 | 820 | 313 | 17763 | 3.88E−14 |
| | | subtype | | | | | |
| GOTERM_BP | C3H | regulation of | 31 | 664 | 139 | 13588 | 6.84E−09 |
| | | cytokine | | | | | |
| | | production | | | | | |
| GOTERM_BP | C3H | cell activation | 50 | 664 | 246 | 13588 | 2.35E−14 |
| GOTERM_BP | C3H | leukocyte | 47 | 664 | 219 | 13588 | 2.73E−14 |
| | | activation | | | | | |
| GOTERM_BP | C3H | lymphocyte | 36 | 664 | 191 | 13588 | 1.64E−08 |
| | | activation | | | | | |
| GOTERM_BP | C3H | T cell activation | 24 | 664 | 116 | 13588 | 1.29E−05 |
| GOTERM_BP | C3H | chemotaxis | 23 | 664 | 109 | 13588 | 1.94E−05 |
| GOTERM_BP | C3H | phagocytosis | 17 | 664 | 49 | 13588 | 1.14E−06 |
| GOTERM_BP | C3H | cell | 33 | 664 | 247 | 13588 | 7.51E−04 |
| | | proliferation | | | | | |
| GOTERM_CC | C3H | plasma | 132 | 655 | 1633 | 12504 | 2.60E−04 |
| | | membrane part | | | | | |
| SP_PIR_KEYWORDS | C3H | collagen | 22 | 824 | 84 | 17854 | 1.97E−07 |
| GOTERM_BP | C3H | response to | 23 | 664 | 157 | 13588 | 1.36E−02 |
| | | bacterium | | | | | |
| GOTERM_BP | C3H | positive | 24 | 664 | 136 | 13588 | 2.87E−04 |
| | | regulation of | | | | | |
| | | immune | | | | | |
| | | response | | | | | |

We examined the expression of a set of osteogenic genes used by us earlier (Cell Stem Cell (2017) 20:1-15, Figure S1B) to represent an osteogenic signature (Cell Stem Cell (2017) 20:1-15, Figure S6D) and observed that the mean expression of osteogenic genes (osteogenic signature) was significantly higher in injured C3H hearts compared to uninjured C3H hearts (Cell Stem Cell (2017) 20:1-15, Figure S6E). The osteogenic signature was not higher in injured B6 hearts compared to control uninjured B6 hearts (Cell Stem Cell (2017) 20:1-15, Figure S6E). B6 mouse hearts had only 1 osteogenic gene that was upregulated (Cell Stem Cell (2017) 20:1-15, Figure S6F); in contrast C3H hearts had 11 osteogenic genes upregulated after injury (Cell Stem Cell (2017) 20:1-15, Figure S6G). Runx2, Enpp1, Col1a1, and Fibronectin were upregulated genes that are well recognized to regulate osteogenesis in the skeleton (Cell Stem Cell (2017) 20:1-15, FIG. 11G).

Calcification of the extracellular matrix is critically regulated by the balance of extracellular phosphate (Pi) and pyrophosphate (PPi) (Terkeltaub, 2001). Pyrophosphate is generated at the cell surface by the enzyme ectonucleotide pyrophosphatase/phosphodiesterase-1 (ENPP1) that breaks down ATP to AMP and PPi. Pyrophosphate is well recognized to inhibit calcium hydroxyapatite mineralization (Rutsch et al., 2011) in non-skeletal tissues, but in bone and teeth, pyrophosphate promotes mineralization by serving as a substrate for tissue non specific alkaline phosphatase that hydrolyzes pyrophosphate to generate inorganic phosphate (Terkeltaub, 2006). ENPP1 is expressed in osteoblasts, thought to regulate osteoblast maturation and bone mineralization and animals deficient in ENPP1 have decreased mineralization of long bones (Johnson et al., 2003). Considering the importance of ENPP1 in mineralization of the skeleton, we examined whether ENPP1, a gene identified by us to be differentially expressed (by RNA-seq) between calcific and non-calcified cardiac regions was contributing to ectopic cardiac calcification. We first confirmed our observation and subjected C3H mice and B6 mice to cryo-induced cardiac injury. We observed with qPCR, that injury increased ENPP1 expression in both C3H and B6 mouse hearts (p<0.05, n=6), but the increase in ENPP1 expression after injury was significantly higher in C3H hearts compared to B6 mice that did not exhibit cardiac calcification after injury (p<0.01, n=6) (FIG. 1; Cell Stem Cell (2017) 20:1-15, FIG. 6A). Immunostaining for ENPP1 confirmed that ENPP1 was expressed in uninjured B6 hearts (Cell Stem Cell (2017) 20:1-15, FIG. 6B) and was more abundant following injury (Cell Stem Cell (2017) 20:1-15, FIG. 6C). Compared to uninjured C3H hearts (Cell Stem Cell (2017) 20:1-15, FIG. 6D), ENPP1 expression was markedly increased in injured C3H mouse hearts (Cell Stem Cell (2017) 20:1-15, FIG. 6E). We next determined whether cardiac fibroblasts in the injury region were a source of increased ENPP1 expression. We subjected Col1a2CreERT:R26R^{tdTomato} mice hearts to cardiac injury and observed abundant expression of ENPP1 by tdTomato labeled cardiac fibroblasts in calcified regions. (Cell Stem Cell (2017) 20:1-15, FIG. 6F-H). Extracellular pyrophosphate generated by ENPP1 can induce mineralization of tissues by precipitating out as calcium pyrophosphate dihydrate (CPPD) or serving as a substrate for phosphate generation and formation of calcium hydroxyapatite. To distinguish between these two, we performed Raman spectroscopy (Chen et al., 2009) and observed that the myocardial calcific deposits comprised calcium hydroxyapatite and not pyrophosphate dihydrate (Cell Stem Cell (2017) 20:1-15, FIG. 61). PPi generated by ENPP1 can be hydrolyzed by tissue non-specific alkaline phosphatase (TNAP) to Pi. The heart is known to express TNAP and we confirmed that both injured and uninjured cardiac tissue is rich in tissue non-specific alkaline phosphatase (TNAP) (Cell Stem Cell (2017) 20:1-15, Figure S7A,B). We measured gene expression and enzymatic activity of alkaline phosphatase and observed abundant expression and activity although TNAP gene expression or activity did not change following injury (Cell Stem Cell (2017) 20:1-15, Figure S7C,D). Biochemical measurements confirmed significantly higher phosphate levels in calcified regions (Cell Stem Cell (2017) 20:1-15, FIG. 6J). Mice that did not exhibit post injury cardiac calcification, in contrast, showed a decrease in phosphate levels in the injured region (Cell Stem Cell (2017) 20:1-15, FIG. 6J), although it is difficult to ascertain from our study whether this contributes to protection from calcification or a consequence of not exhibiting calcification.

We next investigated whether the ENPP1-PPi-Pi axis could be targeted to decrease ectopic cardiac calcification. To address this question, we injected a small molecule inhibitor of ENPP1 (SYL-001) that has been used previously to antagonize ENPP1 in the heart (Sassi et al., 2014) (Cell Stem Cell (2017) 20:1-15, FIG. 6K-N). ENPP1 inhibitor (delivered via continuous infusion) or vehicle was administered for 2 days prior to cardiac injury and for 5 days after injury. Compared to vehicle injected control animals (Cell Stem Cell (2017) 20:1-15, FIG. 6K), animals that received the ENPP1 inhibitor, SYL-001 had decreased post injury cardiac calcification evident on gross inspection (Cell Stem Cell (2017) 20:1-15, FIG. 6L). Micro CT along with 3D reconstruction (Cell Stem Cell (2017) 20:1-15, FIG. 6M,N) showed a 42% decrease in calcific deposits in animals that received the ENPP1 inhibitor (Cell Stem Cell (2017) 20:1-15, FIG. 6N) compared to vehicle treated animals (Cell Stem Cell (2017) 20:1-15, FIG. 6M). To strengthen the evidence that ENPP1 contributes to calcification, we employed another small molecule inhibitor of ENPP1 (ARL67156) (Cote et al., 2012); (Levesque et al., 2007). ARL67156 was administered in an identical manner continuously via a mini pump. ARL67156 similar to SYL-001 significantly inhibited calcification (Cell Stem Cell (2017) 20:1-15, FIG. 60) compared to vehicle injected controls (Cell Stem Cell (2017) 20:1-15, FIG. 6K). A CT scan demonstrated 85% decrease in calcification (Cell Stem Cell (2017) 20:1-15, FIG. 6P). Biochemical measurements demonstrated a significant 35% (p<0.05) and 79% (p<0.05) reduction in calcium deposits in the injured hearts of mice that received the ENPP1 inhibitors SYL-001 or ARL67156 respectively (Cell Stem Cell (2017) 20:1-15, FIG. 6Q).

Bisphosphonates are compounds structurally similar to pyrophosphate where two phosphate moieties are joined by a non-hydrolysable carbon bond rather than an oxygen bond as in pyrophosphate. Although primarily used for their anti-resorptive effect on bone, first generation bisphosphonates such as etidronate can bind to calcium hydroxyapatite in sites of active bone remodeling, and as they are not hydrolysable, prevent further bone mineralization (Drake et al., 2008). In this manner they serve as functional antagonists of the ENPP1-PPi-Pi axis and have been used to decrease ectopic vascular calcification in rodent models of kidney disease (Lomashvili et al., 2009). We investigated whether bisphosphonates could antagonize mineralization in the injured heart. We administered etidronate one day prior to cardiac cryo injury and then daily till the hearts were harvested. In contrast to vehicle injected controls (Cell Stem Cell (2017) 20:1-15, FIG. 6R,S), etidronate completely rescued the calcific phenotype and no calcification was seen on gross inspection or on CT scans (Cell Stem Cell (2017) 20:1-15, FIG. 6T,U). However, etidronate, when administered after the development of calcification, did not reverse or decrease the amount of deposited calcium [data not shown].

We next investigated the physiologic significance of inhibiting cardiac calcification on cardiac function. In a subset of C3H animals, we performed echocardiography to determine cardiac function prior to cardiac cryo-injury and following administration of etidronate. Inhibition of calcification by etidronate was associated with significant preservation of post injury cardiac function (Cell Stem Cell (2017) 20:1-15, FIG. 7A-F). Echocardiography demonstrated better systolic function in injured animals that received etidronate (Cell Stem Cell (2017) 20:1-15, FIG. 7A,B and Table S1).

The left ventricular end diastolic diameter (LVEDD) and end systolic diameter (LVESD) were significantly decreased post injury in etidronate injected animals compared to vehicle treated control animals (Cell Stem Cell (2017) 20:1-15, FIG. 7C,D). Ejection fraction (Cell Stem Cell (2017) 20:1-15, FIG. 7E) and fractional shortening (Cell Stem Cell (2017) 20:1-15, FIG. 7F) were substantially better following injury in animals where calcification was inhibited with etidronate. Etidronate had no effect on sham injured hearts (Cell Stem Cell (2017) 20:1-15, FIG. 7C-F). Taken together, these experiments demonstrate the potential of targeting the ENPP1-PPi-Pi axis for inhibiting ectopic cardiac calcification and augmenting cardiac function.

### Discussion

Cell plasticity is known to play an important physiological role during development and wound healing (Nieto et al., 2016). Mesenchymal stromal cells from different organs have been shown to be capable of inducing calcification in vitro (Ronchetti et al., 2013). Our report suggests that aberrant plasticity of cardiac fibroblasts after injury drive them towards an osteogenic phenotype inducing mineralization of the cardiac extracellular matrix in vivo. The results described here broadly fulfill the Koch's postulates (Evans, 1976) in implicating the fibroblast as a contributor to cardiac calcification i.e. (i) presence of cardiac fibroblasts expressing osteoblast markers in models of cardiac calcification but not in control hearts (ii) induction of calcific phenotype following implantation into another host and (iii) identification of the labeled cardiac fibroblast from calcific lesions of the host animal.

The animal models used are clinically germane to heart calcification in humans. Human cardiac calcification is most often seen after various types of cardiac injury (ischemic, viral, toxic) and thus the use of different modalities of cardiac injury in mice to elicit the phenotype. Moreover, all patients after injury do not develop cardiac calcification and hence the use of different strains of mice to determine a mechanism that is differentially regulated in calcific versus non-calcific hearts.

Our data points to the role of ENPP1 that is differentially expressed in fibroblasts of hearts developing post injury calcification. Osteoblasts express ENPP1 and ENPP1 mediated generation of PPi in bone augments mineralization via hydrolysis of PPi to generate Pi and subsequent hydroxyapatite formation. Our data suggests that similar mechanisms are likely at play in regulating ectopic cardiac calcification. Administration of small molecules that inhibit ENPP1, or a bisphosphonate led to significantly decreased ectopic cardiac calcification and preservation of post injury cardiac function. Pathologic heart calcification is a physiologically important consequence of clinical and subclinical heart injury. Our study identifies the cardiac fibroblast and the ENPP1-PPi-Pi axis as potential cellular and pharmacological targets for treating this pathologic condition.

### REFERENCES

Abdallah, B. M., Ditzel, N., and Kassem, M. (2008). Assessment of bone formation capacity using in vivo transplantation assays: procedure and tissue analysis. Methods Mol Biol 455, 89-100.
Acharya, A., Baek, S. T., Banfi, S., Eskiocak, B., and Tallquist, M. D. (2011). Efficient inducible Cre-mediated recombination in Tcf21 cell lineages in the heart and kidney. Genesis 49, 870-877.
Aherrahrou, Z., Axtner, S. B., Kaczmarek, P. M., Jurat, A., Korff, S., Doehring, L. C., Weichenhan, D., Katus, H. A., and Ivandic, B. T. (2004). A locus on chromosome 7 determines dramatic up-regulation of osteopontin in dystrophic cardiac calcification in mice. Am J Pathol 164, 1379-1387.
Buttitta, L. A., and Edgar, B. A. (2007). Mechanisms controlling cell cycle exit upon terminal differentiation. Curr Opin Cell Biol 19, 697-704.
Chen, C., Uludag, H., Wang, Z., and Jiang, H. (2012). Noggin suppression decreases BMP-2-induced osteogenesis of human bone marrow-derived mesenchymal stem cells in vitro. J Cell Biochem 113, 3672-3680.
Chen, K. H., Li, M. J., Cheng, W. T., Balic-Zunic, T., and Lin, S. Y. (2009). Identification of monoclinic calcium pyrophosphate dihydrate and hydroxyapatite in human sclera using Raman microspectroscopy. Int J Exp Pathol 90, 74-78.
Choi, Y. A., Lim, J., Kim, K. M., Acharya, B., Cho, J. Y., Bae, Y. C., Shin, H. I., Kim, S. Y., and Park, E. K. (2010). Secretome analysis of human BMSCs and identification of SMOC1 as an important ECM protein in osteoblast differentiation. J Proteome Res 9, 2946-2956.
Chong, J. J., Chandrakanthan, V., Xaymardan, M., Asli, N. S., Li, J., Ahmed, I., Heffernan, C., Menon, M. K., Scarlett, C. J., Rashidianfar, A., et al. (2011). Adult cardiac-resident MSC-like stem cells with a proepicardial origin. Cell Stem Cell 9, 527-540.
Cote, N., El Husseini, D., Pepin, A., Bouvet, C., Gilbert, L. A., Audet, A., Fournier, D., Pibarot, P., Moreau, P., and Mathieu, P. (2012). Inhibition of ectonucleotidase with ARL67156 prevents the development of calcific aortic valve disease in warfarin-treated rats. Eur J Pharmacol 689, 139-146.
Crisan, M., Yap, S., Casteilla, L., Chen, C. W., Corselli, M., Park, T. S., Andriolo, G., Sun, B., Zheng, B., Zhang, L., et al. (2008). A perivascular origin for mesenchymal stem cells in multiple human organs. Cell Stem Cell 3, 301-313.
Czernin, J., Satyamurthy, N., and Schiepers, C. (2010). Molecular mechanisms of bone 18F—NaF deposition. J Nucl Med 51, 1826-1829.
Demer, L. L., and Tintut, Y. (2014). Inflammatory, metabolic, and genetic mechanisms of vascular calcification. Arterioscler Thromb Vasc Biol 34, 715-723.
Drake, M. T., Clarke, B. L., and Khosla, S. (2008). Bisphosphonates: mechanism of action and role in clinical practice. Mayo Clin Proc 83, 1032-1045.
Evans, A. S. (1976). Causation and disease: the Henle-Koch postulates revisited. Yale J Biol Med 49, 175-195.
Glass, A. M., Coombs, W., and Taffet, S. M. (2013). Spontaneous cardiac calcinosis in BALB/cByJ mice. Comp Med 63, 29-37.
Graneli, C., Thorfve, A., Ruetschi, U., Brisby, H., Thomsen, P., Lindahl, A., and Karlsson, C. (2014). Novel markers of osteogenic and adipogenic differentiation of human bone marrow stromal cells identified using a quantitative proteomics approach. Stem Cell Res 12, 153-165.
Harkness, L., Mahmood, A., Ditzel, N., Abdallah, B. M., Nygaard, J. V., and Kassem, M. (2011). Selective isolation and differentiation of a stromal population of human embryonic stem cells with osteogenic potential. Bone 48, 231-241.
Hoshiba, T., Kawazoe, N., Tateishi, T., and Chen, G. (2009). Development of stepwise osteogenesis-mimicking matrices for the regulation of mesenchymal stem cell functions. J Biol Chem 284, 31164-31173.
Ivandic, B. T., Qiao, J. H., Machleder, D., Liao, F., Drake, T. A., and Lusis, A. J. (1996). A locus on chromosome 7 determines myocardial cell necrosis and calcification (dystrophic cardiac calcinosis) in mice. Proc Natl Acad Sci USA 93, 5483-5488.
Jaiswal, N., Haynesworth, S. E., Caplan, A. I., and Bruder, S. P. (1997). Osteogenic differentiation of purified, culture-expanded human mesenchymal stem cells in vitro. J Cell Biochem 64, 295-312.
Johnson, K., Goding, J., Van Etten, D., Sali, A., Hu, S. I., Farley, D., Krug, H., Hessle, L., Millan, J. L., and Terkeltaub, R. (2003). Linked deficiencies in extracellular PP(i) and osteopontin mediate pathologic calcification associated with defective PC-1 and ANK expression. J Bone Miner Res 18, 994-1004.
Kanisicak, O., Khalil, H., Ivey, M. J., Karch, J., Maliken, B. D., Correll, R. N., Brody, M. J., SC, J. L., Aronow, B. J., Tallquist, M. D., et al. (2016). Genetic lineage tracing defines myofibroblast origin and function in the injured heart. Nat Commun 7, 12260.
Korff, S., Riechert, N., Schoensiegel, F., Weichenhan, D., Autschbach, F., Katus, H. A., and Ivandic, B. T. (2006). Calcification of myocardial necrosis is common in mice. Virchows Arch 448, 630-638.
Len, A., Kajstura, J., and Anversa, P. (2005). Cardiac Stem Cells and Mechanisms of Myocardial Regeneration. Physiol Rev 85, 1373-1416.
Lev, M. (1964). Anatomic Basis for Atrioventricular Block. Am J Med 37, 742-748.
Levesque, S. A., Lavoie, E. G., Lecka, J., Bigonnesse, F., and Sevigny, J. (2007). Specificity of the ecto-ATPase inhibitor ARL 67156 on human and mouse ectonucleotidases. Br J Pharmacol 152, 141-150.
Liu, C., Gu, S., Sun, C., Ye, W., Song, Z., Zhang, Y., and Chen, Y. (2013). FGF signaling sustains the odontogenic fate of dental mesenchyme by suppressing beta-catenin signaling. Development 140, 4375-4385.
Lomashvili, K. A., Monier-Faugere, M. C., Wang, X., Malluche, H. H., and O'Neill, W. C. (2009). Effect of bisphosphonates on vascular calcification and bone metabolism in experimental renal failure. Kidney Int 75, 617-625.
Miguez, P. A., Terajima, M., Nagaoka, H., Ferreira, J. A., Braswell, K., Ko, C. C., and Yamauchi, M. (2014). Recombinant biglycan promotes bone morphogenetic protein-induced osteogenesis. J Dent Res 93, 406-411.
Murray, I. R., Baily, J. E., Chen, W. C., Dar, A., Gonzalez, Z. N., Jensen, A. R., Petrigliano, F. A., Deb, A., and Henderson, N.C. (2016). Skeletal and cardiac muscle pericytes: Functions and therapeutic potential. Pharmacol Ther.
Nieto, M. A., Huang, R. Y., Jackson, R. A., and Thiery, J. P. (2016). Emt: 2016. Cell 166, 21-45.
Nora, C. C., Camassola, M., Bellagamba, B., Ikuta, N., Christoff, A. P., Meirelles Lda, S., Ayres, R., Margis, R., and Nardi, N. B. (2012). Molecular analysis of the differentiation potential of murine mesenchymal stem cells from tissues of endodermal or mesodermal origin. Stem Cells Dev 21, 1761-1768.
Olivares-Navarrete, R., Hyzy, S. L., Park, J. H., Dunn, G. R., Haithcock, D. A., Wasilewski, C. E., Boyan, B. D., and Schwartz, Z. (2011). Mediation of osteogenic differentiation of human mesenchymal stem cells on titanium surfaces by a Wnt-integrin feedback loop. Biomaterials 32, 6399-6411.
Pugashetti, R., Shinkai, K., Ruben, B. S., Grossman, M. E., Maldonado, J., and Fox, L. P. (2011). Calcium may preferentially deposit in areas of elastic tissue damage. J Am Acad Dermatol 64, 296-301.
Qian, L., Huang, Y., Spencer, C. I., Foley, A., Vedantham, V., Liu, L., Conway, S. J., Fu, J. D., and Srivastava, D. (2012). In vivo reprogramming of murine cardiac fibroblasts into induced cardiomyocytes. Nature 485, 593-598.
Romanoski, C. E., Lee, S., Kim, M. J., Ingram-Drake, L., Plaisier, C. L., Yordanova, R., Tilford, C., Guan, B., He, A., Gargalovic, P. S., et al. (2010). Systems genetics analysis of gene-by-environment interactions in human cells. Am J Hum Genet 86, 399-410.
Ronchetti, I., Boraldi, F., Annovi, G., Cianciulli, P., and Quaglino, D. (2013). Fibroblast involvement in soft connective tissue calcification. Front Genet 4, 22.
Rostand, S. G., Sanders, C., Kirk, K. A., Rutsky, E. A., and Fraser, R. G. (1988). Myocardial calcification and cardiac dysfunction in chronic renal failure. Am J Med 85, 651-657.
Rutsch, F., Nitschke, Y., and Terkeltaub, R. (2011). Genetics in arterial calcification: pieces of a puzzle and cogs in a wheel. Circ Res 109, 578-592.
Sassi, Y., Ahles, A., Truong, D. J., Baqi, Y., Lee, S. Y., Husse, B., Hulot, J. S., Foinquinos, A., Thum, T., Muller, C. E., et al. (2014). Cardiac myocyte-secreted cAMP exerts paracrine action via adenosine receptor activation. J Clin Invest 124, 5385-5397.
Shackley, B. S., Nguyen, T. P., Shivkumar, K., Finn, P. J., and Fishbein, M. C. (2011). Idiopathic massive myocardial calcification: a case report and review of the literature. Cardiovasc Pathol 20, e79-83.
Simchen, M. J., Toi, A., Silver, M., Smith, C. R., Hornberger, L. K., Taylor, G., and Chitayat, D. (2006). Fetal cardiac calcifications: report of four prenatally diagnosed cases and review of the literature. Ultrasound Obstet Gynecol 27, 325-330.
Song, K., Nam, Y. J., Luo, X., Qi, X., Tan, W., Huang, G. N., Acharya, A., Smith, C. L., Tallquist, M. D., Neilson, E. G., et al. (2012). Heart repair by reprogramming non-myocytes with cardiac transcription factors. Nature 485, 599-604.
Sparks, L. L., Rosenau, W., Macalpin, R. N., Daane, T. A., and Li, C. H. (1955). Production of dystrophic calcification of cardiac muscle in mice by hydrocortisone. Nature 176, 503-504.
Stallion, A., Rafferty, J. F., Warner, B. W., Ziegler, M. M., and Ryckman, F. C. (1994). Myocardial calcification: a predictor of poor outcome for myocarditis treated with extracorporeal life support. J Pediatr Surg 29, 492-494.
Terkeltaub, R. (2006). Physiologic and pathologic functions of the NPP nucleotide pyrophosphatase/phosphodiesterase family focusing on NPP1 in calcification. Purinergic Signal 2, 371-377.
Terkeltaub, R. A. (2001). Inorganic pyrophosphate generation and disposition in pathophysiology. Am J Physiol Cell Physiol 281, C1-C11.
Ubil, E., Duan, J., Pillai, I. C., Rosa-Garrido, M., Wu, Y., Bargiacchi, F., Lu, Y., Stanbouly, S., Huang, J., Rojas, M., et al. (2014). Mesenchymal-endothelial transition contributes to cardiac neovascularization. Nature 514, 585-590.
Zheng, B., Zhang, Z., Black, C. M., de Crombrugghe, B., and Denton, C. (2002). Ligand-dependent genetic recombination in fibroblasts: a potentially powerful technique for investigating gene function in fibrosis. Am J Pathol 160, 1609-1617.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control. The compounds, synthetic methods, and experimental protocols and results of U.S. application Ser. No. 13/680,582, filed Nov. 19, 2012, are hereby incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing ectopic calcification in a subject, comprising administering an ENPP1 inhibitor.

2. The method of claim 1, wherein the ENPP1 inhibitor is selected from rosmarinic acid, ARL67156, and etidronic acid, or a pharmaceutically acceptable salt and/or prodrug thereof.

3. The method of claim 1, wherein the ENPP1 inhibitor is rosmarinic acid or a pharmaceutically acceptable salt and/or prodrug thereof.

4. The method of claim 1, wherein the ENPP1 inhibitor is ARL67156 or a pharmaceutically acceptable salt and/or prodrug thereof.

5. The method of claim 1, wherein the ENPP1 inhibitor is etidronic acid or a pharmaceutically acceptable salt and/or prodrug thereof.

6. The method of claim 1, wherein the subject has diabetes, kidney disease, heart disease and/or myocardial injury, or vascular disease.

7. The method of claim 6, wherein the subject has heart disease.

8. The method of claim 7, wherein the ectopic calcification is in heart tissue.

9. The method of claim 8, wherein the heart issue is a heart valve.

10. The method of claim 6, wherein the subject has myocardial injury.

11. The method of claim 1, further comprising inhibiting the level of cardiac fibroblast calcification activity.

12. The method of claim 1, wherein the subject has a soft tissue injury or organ injury.

13. The method of claim 1, wherein the subject has pseudoxanthoma elasticum.

14. The method of claim 1, further comprising reducing cellular levels of pyrophosphate.

15. The method of claim 1, wherein rosmarinic acid and ARL67156 are conjointly administered to the subject.

16. The method of claim 1, wherein rosmarinic acid and etidronic acid are conjointly administered to the subject.

17. The method of claim 1, wherein ARL67156 and etidronic acid are conjointly administered to the subject.

18. The method of claim 1, wherein a bisphosphonate is conjointly administered with an ENPP1 inhibitor.

19. The method of claim 18, wherein the bisphosphonate is selected from clondrate, tiludronate, pamidronate, neridronate, olpadronate, alendronate, ibandronate, risedronate, and zoledronate.
